(19) United States
(12) Patent Application Publication
(10) Pub. No.: US 2006/0286164 A1
(43) Pub. Date: Dec. 21, 2006

(54) Pharmaceutical composition containing a stable and clear solution of anti-inflammatory drug in soft gelatin capsule and process for producing the same

(76) Inventors: Venkat Subramanian Iyer, Karnataka (IN); Shivaraj Basavaraj Katageri, Karnataka (IN)

Correspondence Address: NEIFELD IP LAW, PC, 4813-B EISENHOWER AVENUE, ALEXANDRIA, VA, 22304, US

(21) Appl. No.: 10/545,601

(22) PCT Filed: Jun. 7, 2004
(86) PCT No.: PCT/IN04/00158

§ 371 Date: Aug. 16, 2005

(51) Int. Cl. A61K 31/192; A61K 9/64; A61K 9/14

(52) U.S. Cl. 424/456; 424/486; 514/569

(57) ABSTRACT

Disclosed herein is a pharmaceutical composition comprising a soft gelatin capsule containing a clear and stable solution of sodium dihydratate salt of ibuprofen by employing a system of solubilizer, co-solubilizer and antioxidants.

### FIELD OF THE INVENTION

In general, this invention relates to a pharmaceutical composition containing non-steroidal anti-inflammatory agent. More particularly the present invention provides for a pharmaceutical composition containing substantially stable and clear solution of Ibuprofen Sodium dihydrate encapsulated into soft gelatin capsules and process for producing the same.

### BACKGROUND OF THE INVENTION

Ibuprofen is chemically 2-(4-Isobutylphenyl)Propionic acid, practically insoluble in water. It is a very effective Non steroidal anti-inflammatory drug (NSAID), analgesic (pain reliever), and antipyretic (fever reducer). NSAID is the general term used for a group of drugs that are effective in reducing inflammation and pain.

The major problem of developing pharmaceutical formulations is to address the poor solubility in water and unpleasant taste of pharmaceutical drugs and the subsequent poor bioavailability. To solve these problems different approaches are taken like developing suspensions, solubilizing in organic solvents, using salts of the drugs, developing prodrugs and using different types of drug delivery systems.

Ibuprofen (racemic mixture) or (S)-(+)-Ibuprofen are not soluble in water, but the sodium dihydrate salt of Ibuprofen is freely soluble in water. It has been found that the usual sodium, calcium and magnesium salts of Ibuprofen also have a discernible disagreeable taste like the acidic drug Ibuprofen.

The freely water-soluble Ibuprofen Sodium dihydrate makes a good candidate for preparing a pharmaceutical formulation having better bioavailability. The disagreeable taste of the pharmaceutical agent is taken care by using different drug delivery systems.

One such drug delivery system is the soft gelatin, or softgel capsule. The patient compliance is also improved if soft gelatin capsule is used for drug administration, because of its soft, elastic character making it easier to swallow compared to conventional dosage forms like tablets and hard gelatin capsules.

Compared to direct oral liquid administration, the encapsulation of the liquid in soft gelatin capsule has benefits of non-spillage, encapsulation of active agents having non-agreeable taste, provide unit dose of medicament avoiding the need of measuring the liquid medicament. Soft gels dissolve rapidly and release the liquid medicament for ready absorption.

Filled one-piece soft gel capsules have been widely known and used for many years and for a variety of purposes. Because soft gel capsules have properties, which are different from conventional two-piece hard shell capsules, the soft gel capsules are capable of retaining liquid fill material. Another drug delivery issue is content uniformity. If a formulation is a true solution, content uniformity can be achieved. The active ingredient may be completely dissolved in a softgel formulation.

In general, not all liquids are suitable as vehicles or carriers for inclusion in soft gel capsules. For example, water, propylene glycol, glycerin, low molecular weight alcohols, ketones, acids, amines and esters cannot be used as a carrier in soft gel capsules by themselves since they interact with the gel and, if present, they can only be present in relatively small amounts.

Another limitation associated with soft gel capsules is the inability to incorporate a single dose of the pharmaceutically active ingredient in solution, in an acceptable fill volume. Often, it is difficult to dissolve the pharmaceutically active ingredient in a volume of solvent small enough to incorporate in a soft gel capsule, which delivers the desired dosage amount, is economically appropriate and comfortable to ingest by the patient. Developing solvent systems for pharmaceutically active ingredients that neither significantly interact with the active ingredient nor with the soft gel casing itself, has proven a difficult art.

### RELATED ART

U.S. Pat. No. 6,525,214 to Armitage, et al., discloses the use of S(−)sodium 2-(4-isobutylphenyl)propionate in pharmaceutical compositions and the process to prepare S(−)sodium 2-(4-isobutylphenyl)propionate. Different dosage forms like solid dosage form, oral liquid compositions and compositions for topical administration are disclosed in this Patent.

U.S. Pat. No. 5,510,385 to Stroppolo, et al., discloses a pharmaceutical composition comprising a salt of S(+)-Ibuprofen with a basic amino acid selected between L-arginine and L-lysine. The solid dosage forms disclosed are powders, granulates, tablets and capsules. Use of sweetening agents, flavoring agents, diluents, disintegrating agents, lubricating agents (Polyethylene glycol) and thickening agents (Polyvinylalcohol, Polyvinylpyrrolidone) is disclosed.

U.S. Pat. No. 5,696,165 to Armitage, et al., discloses solid or semi-solid pharmaceutical compositions comprising S(−)sodium 2-(4-isobutyl)propionate having an enantiomeric purity of at least 90% as the sole form of 2-(4-isobutylphenyl)propionate. Also disclosed is the use of S(−)sodium 2-(4-isobutyl)propionate as dihydrate form. The dosage forms disclosed are tablets, capsules, cream, ointment, gel, poultice or patch.

U.S. Pat. No. 6,242,000 to Armitage, et al., discloses a pharmaceutical composition comprising S(−)sodium 2-(4-isobutylphenyl)propionate dihydrate having an enantiomeric purity of at least 90%; and a pharmaceutically acceptable carrier. The dosage forms disclosed are tablets, granules, capsules, liquid dosage forms, gel, suppository, etc.

U.S. Pat. No. 5,541,227 to Loew, et al., discloses an Ibuprofen containing medicament which contains Ibuprofen only in the (S)-(+)-form in a tablet dosage form which permits reduction of the quantity of active ingredient and the size of the tablet or dragee. This patent discloses solubilised form of either Ibuprofen or Dexibuprofen.

U.S. Pat. No. 4,690,823 to Lohner, et al., discloses the Ibuprofen containing soft gelatin capsules and process for preparing the same. This particular invention makes use of about 70 to 85% by weight of polyoxyethylene-polyoxypropylene polymer or mixture of from 30 to 76% parts by weight of polyalkylene glycol and from 7 to 40% by weight of surfactants to dissolve about 15 to 30% parts by weight of Ibuprofen.

U.S. Pat. No. 6,294,192 to Patel, et al., discloses a triglyceride free composition of hydrophobic therapeutic agents and a carrier, where the composition forms a clear, aqueous dispersion of the surfactants containing the therapeutic agent upon dilution with an aqueous solvent. The carrier of the said composition is made of a hydrophilic surfactant and a hydrophobic surfactant. Ibuprofen is disclosed as one of the therapeutic agents among the hydrophobic therapeutic agents. Ethyl alcohol and Transcutol are disclosed as solubilizers among a group of solubilizers used in the composition. Also disclosed herein is the encapsulation of the composition in a hard or soft gelatin capsule.

U.S. Pat. No. 6,267,985 to Chen, et al., discloses unique pharmaceutical compositions, which form clear aqueous dispersions upon mixing with an aqueous solution. Disclosed herein is the compositions including triglycerides and a combination of surfactants that can solubilize therapeutically effective amounts of therapeutic agents in homogeneous, single-phase systems, which are thermodynamically stable and optically clear. Transcutol is used as one of the solubilizers. Ibuprofen is disclosed as one of the therapeutic agents. The pharmaceutical composition can be preconcentrate in a liquid, semi-solid or solid form or as aqueous or organic diluted preconcentrate. Dosage form disclosed is not limited.

U.S. Pat. No. 5,019,563 to Huntel, et al., discloses complexes of beta-cyclodextrin with various salts of Ibuprofen in which the molar ratios of Ibuprofen to beta-cyclodextrin are within the range of from 1:0.2 to 1:0.75. The preferred salt of Ibuprofen is the sodium salt. The compositions disclosed in this invention are granules or tablets, which further include an amount of a pharmaceutically acceptable acid salt such as sodium citrate or a buffer system such that when the composition is added to water, the pH of the resultant solution is between 6.0 and 8.0.

U.S. Pat. No. 6,221,391 to Rouffer, teaches the self-emulsifying Ibuprofen solution in soft gelatin capsule for use therewith. Polyoxyethylene castor oil derivatives have been used in this formulation to provide self emulsifying properties to the formulation.

U.S. Pat. No. 6,251,426 to Gullapalli, discloses Ibuprofen containing softgels wherein Ibuprofen is present as free acid form and softgel capsules are comprised of a gelatin sheath enclosing such fill formulations. Such formulations are prepared by dissolving more than 30% of Ibuprofen in free acid form in polyethylene glycol and at least 10% by weight of polyvinylpyrrolidone having an average molecular weight of from about 2,000 to about 54,000. This formulation may make use of surfactants to increase bioavailability of Ibuprofen.

U.S. Pat. No. 5,071,643 to Yu, et al., discloses the use of a water based solvent system for enhancing the solubility of an acidic, basic or amphoteric pharmaceutical agent, such as Ibuprofen, to produce a highly concentrated solution suitable for encapsulation. The solvent system includes polyethylene glycol containing 0.2 to 1.0 mole equivalents of an ionizing agent per mole equivalent of pharmaceutical agent and 1 to 20% water. This water based solvent system provides for a highly concentrated solution capable of encapsulation into a small enough vessel, such as a softgel capsule, to permit easy swallowing and to provide a pharmaceutically effective dose of a pharmaceutical agent such as Ibuprofen.

U.S. Pat. No. 6,436,430 to Mulye, is directed to a pharmaceutical composition comprising a pharmaceutically effective amount of a lipophilic drug in association with a pharmaceutical carrier, said carrier comprising a lipophilic drug solubilizing effective amount of a propylene glycol monoester of C6 to C18 fatty acid having at least 60% by weight monoester based on the total weight of the propylene glycol ester and a non-ionic surfactant. The lipophilic drugs disclosed in this invention include Ibuprofen, Naproxen and Paclitaxel.

None of the above-cited prior arts disclose specifically, the utilization of Ibuprofen Sodium dihydrate as source of Ibuprofen.

### SUMMARY OF THE INVENTION

In order to provide better patient compliance, embodiments of the present invention include a pharmaceutical composition comprised in a soft gelatin capsule, the composition containing a clear and stable solution of sodium dihydratate salt of ibuprofen by employing a system of solubilizer and co-solubilizer.

In one particular embodiment, the present invention provides for soft gelatin capsules of a pharmaceutical composition comprising about 30.0% to 35.0% by weight of Ibuprofen sodium dihydrate, about 55.0% to 65.0% by weight of Oleic acid as a solubiliser, about 0.6% to 1.2% by weight of Polyvinylpyrrolidone as an agent to improve solubility, about 4.0% to 5.0% by weight of Propylene glycol as a co-solubilizing agent and about 0.3% to 0.7% by weight of Vitamin E as an antioxidant.

In another embodiment, the present invention provides for soft gelatin capsules of a pharmaceutical composition comprising about 20.0% to 22.0% by weight of Ibuprofen sodium dihydrate, about 70.0% to 75.0% by weight of Diethylene glycol monoethyl ether as a solubiliser, about 0.8% to 1.2% by weight of Polyvinylpyrrolidone as an agent to improve solubility and about 3.0% to 5.0% by weight of Propylene glycol as a co-solubilizing agent.

In yet another embodiment, the present invention provides for soft gelatin capsules of a pharmaceutical composition comprising about 28.0% to 30.0% by weight of Ibuprofen sodium dihydrate, about 9.0% to 11.0% by weight of Ethyl alcohol as a solubiliser, about 4.0% to 6.0% by weight of Propylene glycol as an agent to improve solubility and about 50.0% to 60.0% by weight of Polyethylene glycol 400 as a co-solubilizing agent.

In accordance with still another preferred embodiment, there is provided a process for producing soft gelatin capsule containing a substantially stable and clear solution of sodium dihydrate salt of ibuprofen, the process comprising premixing the solubilizer and co-solubilizer, followed by addition of Ibuprofen sodium dihydrate and further mixing, and disposing the resultant into soft gelatin capsules.

In accordance with still another preferred embodiment, there is provided a process for producing soft gelatin capsule containing a substantially stable and clear solution of sodium dihydrate salt of ibuprofen, the process comprising of solubilizing Polyvinylpyrrolidone in Propylene glycol, adding Oleic acid to it, followed by addition of Ibuprofen sodium dihydrate and Vitamin E and further mixing and encapsulating the same into soft gelatin capsules.

In accordance with still another preferred embodiment, there is provided a process for producing soft gelatin capsule containing a substantially stable and clear solution of sodium dihydrate salt of ibuprofen, the process comprising of solubilizing Polyvinylpyrrolidone in Propylene glycol, adding Diethylene glycol monoethyl ether to it, followed by addition of Ibuprofen sodium dihydrate and further mixing, and encapsulating the same into soft gelatin capsules.

In accordance with still another preferred embodiment, there is provided a process for producing soft gelatin capsule containing a substantially stable and clear solution of sodium dihydrate salt of ibuprofen, the process comprising of solubilizing Ibuprofen sodium dihydrate in a mixture of Ethyl alcohol, Propylene glycol and Polyethylene glycol and further mixing, and encapsulating the same into soft gelatin capsules.

In still another preferred embodiment, there is provided shell composition of soft gelatin capsule comprising of about 45.0% by weight of Gelatin, about 20% by weight of Glycerin, and about 35% by weight of Purified water.

In still another preferred embodiment, there is provided shell composition of a soft gelatin capsule comprising of about 45.0% by weight of Gelatin, about 14% by weight of Glycerin, about 9% by weight of Sorbitol solution, and about 32% by weight of Purified water.

In still another preferred embodiment, there is provided shell composition of a soft gelatin capsule comprising of about 50.0% by weight of Gelatin, about 18% by weight of Glycerin, and about 32% by weight of Purified water.

In still another preferred embodiment, there is provided shell composition of a soft gelatin capsule comprising of about 40.0% by weight of Gelatin, about 16% by weight of Glycerin, and about 44% by weight of Purified water.

In still another preferred embodiment, there is provided shell composition of a soft gelatin capsule comprising of about 48.0% by weight of Gelatin, about 20% by weight of Anidrisorb 85/70, and about 32% by weight of Purified water.

In still another preferred embodiment, there is provided shell composition of a soft gelatin capsule comprising of about 45.0% by weight of Gelatin, about 14% by weight of Glycerin, about 9% by weight of Anidrisorb 85/70 and about 32% by weight of Purified water.

### DETAILED DESCRIPTION OF THE INVENTION

The present invention provides, stable and clear solution of Ibuprofen sodium dihydrate in soft gelatin capsules, which when taken orally release the contents of capsule into media of gastrointestinal tract. Since this formulation contains Ibuprofen sodium dihydrate, which is water soluble, does not precipitate in the contents of gastrointestinal tract, present invention does not necessarily calls for addition of any Surfactant/s in formulation, because of inherent property of Ibuprofen sodium dihydrate being water soluble.

To get a stable and clear solution of Sodium dihydrate salt of Ibuprofen we conducted solubility studies on various solubilizing agents and co-solubilizing agents, including Polyethylene glycols, propylene glycol, Triacetin, Ethyl alcohol, Polyethylene glycol, esters of fatty acids, Propylene glycol esters of fatty acids, Glycerin, Oleic acid etc. separately and mixture thereof. We discovered that a system containing Oleic acid, Propylene glycol and Polyvinylpyrrolidone with Vitamin E, a system containing Diethylene glycol monoethyl ether, Propyleneglycol and Polyvinylpyrrolidone, and a system containing Ethyl alcohol, Propyleneglycol and Polyethylene glycol has resulted in stable and clear solution of Ibuprofen sodium dihydrate suitable for encapsulation into soft gelatin capsules.

Diethylene glycol monoethyl ether is commercially available as Transcutol HP/Transcutol (Gattefosse). Diethylene glycol monoethyl ether solubilizes drugs that are commonly thought to be insoluble or difficult to solubilize. It is soluble in water, ethanol, hexylene glycol and propylene glycol and is partially soluble in vegetable oil. LD50 of Transcutol is 7.5 mg/kg (oral route-rat). (Ref: European pharmacopoeia, Transcutol Product Profile, supplied by M/s Gattefosse—France)

Propylene glycol is used in wide variety of pharmaceutical formulations and is generally regarded as a nontoxic material. It is used as solvent, antimicrobial preservative, disinfectant, humectant, plasticizer, water-miscible cosolvent and stabilizer for vitamins. Propylene Glycol is a clear, colorless, viscous, practically odorless liquid with a sweet, slightly acrid taste resembling glycerin. It is official in British Pharmacopoeia and USP. Propylene glycol is used in a wide variety of pharmaceutical formulations and is generally regarded as a nontoxic material. Based on metabolic and toxicological data, the WHO has set an acceptable daily intake of propylene glycol at up to 25 mg/kg body weight. (Ref: Handbook of Pharmaceutical Excipients, 2nd edition, P. No: 407-408)

Inclusion of Polyethylene Glycol 400 was found to be useful. Polyethylene Glycols can be used to enhance the aqueous solubility or dissolution characteristics of poorly soluble drugs. Polyethylene glycols are also called as Macrogols. Macrogols are relatively stable, non-toxic compounds, which have a range of properties depending on their molecular weight. They are widely used in pharmaceutical manufacturing as water soluble bases for topical preparations and suppositories, as solvents and vehicles, and as solubilising agents, tablet binders, plasticizers in film coating, and tablet lubricants. They have also been reported to have antibacterial properties. (Ref: Martindale, The Complete Drug Reference—33rd edition, P. No. 1630)

Polyvinylpyrrolidone (PVP K-30) has been used in a variety of Pharmaceutical formulations. It has a property of increasing viscosity and an ability to increase solubility of poorly soluble active drugs. It is used in this formulation to enhance the solubility of Ibuprofen sodium dihydrate and to prevent the recrystallisation. (Ref: Handbook of Pharmaceutical Excipients, 2nd edition, P. No: 392)

Ethyl alcohol is also called as Alcohol or Ethanol. Alcohol is a clear, colorless, mobile and volatile liquid with a slight, characteristic odor and burning taste. Alcohol is a powerful solubiliser for the drugs that are commonly thought to be insoluble or difficult to solubilise. Alcohol is miscible with chloroform, ether, glycerin and water. Ethanol and aqueous solutions are widely used in a variety of pharmaceutical formulations and cosmetics. LD50 (Guinea Pig, Oral) is 5.56 g/kg, LD50 (Mouse, Oral) is 7.5 g/kg, LD50 (Rat, Oral) is 7.06 g/kg and LD50 (Rabbit, Oral) is 6.3 g/kg. (Refer: Handbook of Pharmaceutical Excipients, 2nd edition, P. No: 7 to 8)

Oleic acid is chemically (Z)-9-Octadecenoic acid and is used as a vehicle in manufacturing of soft gelatin capsules. Oleic acid is yellowish to pale brown, oily liquid with a characteristic odor. LD50 (mouse, IV)=0.23 g/kg, LD50 (rat, IV)=2.4 mg/kg and LD50 (rat, oral)=74.0 g/kg. Oleic acid is Generally Recognized As Safe (GRAS) and is official in British Pharmacopoeia and United States Pharmacopoeia/National Formulary. (Refer: Handbook of Pharmaceutical Excipients, 2nd edition, P. No: 325)

Vitamin E also called tocopherols is used as anti-oxidants in the formulation. Inclusion of Vitamin E is advisable in the formulations containing the vegetable oils, such as Oleic acid. Vitamin E is practically insoluble in water; freely soluble in acetone, ethanol, ether and vegetable oil. Vitamin E is Generally Recognized As Safe (GRAS) and is official in British Pharmacopoeia, European Pharmacopoeia and United States Pharmacopoeia. (Ref: Handbook of Pharmaceutical Excipients, 2nd edition, P. No: 12 to 13)

Preferred embodiment is further illustrated in the following examples. These examples illustrate particular embodiments of the invention and are not intended to limit the scope of the invention in any way.

### EXAMPLE 1

| S. No. | Ingredients | Quantity (mg) |
|---|---|---|
| 1 | Ibuprofen Sodium Dihydrate | 255.00 |
| 2. | Polyvinylpyrrolidone (PVP K-30) | 6.00 |
| 3. | Propylene glycol | 35.00 |
| 4. | Oleic acid | 450.25 |
| 5. | Vitamin E | 3.75 |
| | Fill weight/capsule | 750.00 |
| | Size | 11 minim Oblong |

Process for Preparation:

Solubilising Polyvinylpyrrolidone in Propylene glycol, adding Oleic acid to it followed by addition of Ibuprofen sodium dihydrate and Vitamin E and further mixing; and disposing the solution into soft gelatin capsules.

### EXAMPLE 2

| S. No. | Ingredients | Quantity (mg) |
|---|---|---|
| 1. | Ibuprofen Sodium Dihydrate | 255.00 |
| 2. | Polyvinylpyrrolidone (PVP K-30) | 12.00 |
| 3. | Propylene glycol | 48.00 |
| 4. | Diethylene glycol monoethyl ether | 885.00 |
| | Fill weight/capsule | 1200.00 |
| | Size | 20 minim Oblong |

Process for Preparation:

Solubilising Polyvinylpyrrolidone in Propylene glycol, adding Diethylene glycol monoethyl ether to it followed by addition of Ibuprofen sodium dihydrate and further mixing; and disposing the solution into soft gelatin capsules.

### EXAMPLE 3

| S. No. | Ingredients | Quantity (mg) |
|---|---|---|
| 1. | Ibuprofen Sodium Dihydrate | 255.00 |
| 2. | Ethyl alcohol | 85.00 |
| 3. | Propylene glycol | 42.50 |
| 4. | Polyethylene glycol | 467.50 |
| | Fill weight/capsule | 850.00 |
| | Size | 14 minim Oblong |

Process for Preparation:

Solubilising Ibuprofen sodium dihydrate in a mixture of Ethyl alcohol, Propylene glycol and Polyethylene glycol and further mixing; and disposing the solution into soft gelatin capsules.

In general, gelatin shell formulations for soft gelatin capsules comprise of gelatin and one or more plasticizer added to adjust the hardness of the capsule. Typical plasticizers include glycerin, sorbitol and Anidrisorb 85/70. One preferred gelatin formulation for the soft gelatin capsules used in accordance with preferred embodiments includes gelatin in the range of about 40% to 50% by weight and a plasticizer in the range of 15% to 25% by weight. Capsule shell may also include other suitable additives, for example coloring agents, which impart specific characteristics such as the look and feel of the capsule and anti-oxidants, opacifiers and etc. FD & C dyes and D & C dyes are examples of pharmaceutically acceptable coloring agents that may be used in the preferred embodiments.

The following examples illustrate the preferred embodiments of soft gelatin shell formulation as used in the present invention. These examples illustrate particular embodiments of the invention and are not intended to limit the scope of the invention in any way.

### EXAMPLE 4

| Ingredient | Percentage by weight |
|---|---|
| Gelatin | 45 |
| Glycerin | 20 |
| Purified water | 35 |

### EXAMPLE 5

| Ingredient | Percentage by weight |
|---|---|
| Gelatin | 45 |
| Glycerin | 14 |
| Sorbitol Solution | 9 |
| Purified water | 32 |

### EXAMPLE 6

| Ingredient | Percentage by weight |
|---|---|
| Gelatin | 50 |
| Glycerin | 18 |
| Purified water | 32 |

### EXAMPLE 7

| Ingredient | Percentage by weight |
|---|---|
| Gelatin | 40 |
| Glycerin | 16 |
| Purified water | 44 |

### EXAMPLE 8

| Ingredient | Percentage by weight |
|---|---|
| Gelatin | 48 |
| Anidrisorb 85/70 | 20 |
| Purified water | 32 |

### EXAMPLE 9

| Ingredient | Percentage by weight |
|---|---|
| Gelatin | 45 |
| Glycerin | 14 |
| Anidrisorb 85/70 | 9 |
| Purified water | 32 |

Certain modifications and improvements of the disclosed invention will occur to those skilled in the art without departing from the scope of invention, which is limited only by the appended claims.

1. A pharmaceutical composition comprising:
a substantially stable and clear solution of an anti-inflammatory drug in a soft gelatin capsule,
wherein the solution comprises:
Sodium dihydrate salt of Ibuprofen;
a solubilizing agent;
a co solubilizing agent;
solubility enhancer; and
an antioxidant.

2-18. (canceled)

19. The pharmaceutical composition according to claim 1, wherein the Sodium dihydrate salt of Ibuprofen is used in the range of about 30.0% to 35.0% by weight.

20. The pharmaceutical composition according to claim 1, wherein the Sodium dihydrate salt of Ibuprofen is used in the range of about 20.0% to 22.0% by weight.

21. The pharmaceutical composition according to claim 1, wherein the Sodium dihydrate salt of Ibuprofen is used in the range of about 28.0% to 30.0% by weight.

22. The pharmaceutical composition according to claim 1, wherein the solubilizing agent is at least one selected from the group comprising Oleic acid, Diethylene glycol monoethyl ether, Ethyl alcohol.

23. The pharmaceutical composition according to claim 22, wherein the Oleic acid is used in the range of about 55.0% to 65.0% by weight.

24. The pharmaceutical composition according to claim 22, wherein the Diethylene glycol monoethyl ether is used in the range of about 70.0% to 75.0% by weight.

25. The pharmaceutical composition according to claim 22, wherein the Ethyl alcohol is used in the range of about 9.0% to 11.0% by weight.

26. The pharmaceutical composition according to claim 1, wherein co-solubilizing agent is at least one selected from Propylene glycol or Polyethylene glycol.

27. The pharmaceutical composition according to claim 26, wherein the Propylene glycol is used in the range of about 4.0% to 5.0% by weight.

28. The pharmaceutical composition according to claim 26, wherein the Propylene glycol is used in the range of about 3.0% to 5.0% by weight.

29. The pharmaceutical composition according to claim 26, wherein the Propylene glycol is used in the range of about 4.0% to 6.0% by weight.

30. The pharmaceutical composition according to claim 26, wherein the Polyethylene glycol is used in the range of about 50.0% to 60.0% by weight.

31. The pharmaceutical composition according to claim 1, wherein solubility enhancer is at least one selected from Polyvinylpyrrolidone or Propylene glycol.

32. The pharmaceutical composition according to claim 31, wherein the Polyvinylpyrrolidone is used in the range of about 0.8% to 1.2% by weight.

33. The pharmaceutical composition according to claim 31, wherein the Propylene glycol is used in the range of about 4.0% to 6.0% by weight.

34. The pharmaceutical composition according to claim 1, wherein the antioxidant is Vitamin E.

35. The pharmaceutical composition according to claim 34, wherein the Vitamin E is used in the range of about 0.3% to 0.7% by weight.

36. An orally administrable pharmaceutical composition according to claim 1, the said soft gelatin capsule having a shell comprising about 45.0% by weight of Gelatin, about 20% by weight of Glycerin, and about 35% by weight of Purified water.

37. An orally administrable pharmaceutical composition according to claim 1, the said soft gelatin capsule having a shell comprising about 45.0% by weight of Gelatin, about 14% by weight of Glycerin, about 9% by weight of Sorbitol solution, and about 32% by weight of Purified water.

38. An orally administrable pharmaceutical composition according to claim 1, the said soft gelatin capsule having a shell comprising about 50.0% by weight of Gelatin, about 18% by weight of Glycerin, and about 32% by weight of Purified water.

39. An orally administrable pharmaceutical composition according to claim 1, the said soft gelatin capsule having a shell comprising about 40.0% by weight of Gelatin, about 16% by weight of Glycerin, and about 44% by weight of Purified water.

40. An orally administrable pharmaceutical composition according to claim 1, the said soft gelatin capsule having a shell comprising about 48.0% by weight of Gelatin, about 20% by weight of Anidrisorb 85/70, and about 32% by weight of Purified water.

41. An orally administrable pharmaceutical composition according to claim 1, the said soft gelatin capsule having a shell comprising about 45.0% by weight of Gelatin, about 14% by weight of Glycerin, about 9% by weight of Anidrisorb 85/70 and about 32% by weight of Purified water.

42. A process for producing pharmaceutical composition as claimed in claim 1, comprising:
adding solubility enhancer to co-solubilizing agent with constant mixing;
adding Ibuprofen sodium dihydrate to the solution with continuous mixing;
adding antioxidant to the resultant, further mixing to get clear solution; and
disposing resulting solution in soft gelatin capsules.

43. The process according to claim 42, wherein the solubility enhancer is preferably Polyvinyl pyrrolidone.

44. The process according to claim 42, wherein the co-solubilizing agent is preferably Propylene glycol.

45. The process according to claim 42, wherein the antioxidant is preferably Vitamin E.

46. A process for producing pharmaceutical composition as claimed in claim 1, comprising:
adding solubility enhancer in co-solubilizing agent with mixing;
adding solubilizing agent to the mixture & further mixing;
adding Ibuprofen sodium dihydrate to the resultant & further mixing; and
disposing resulting solution in soft gelatin capsules.

47. The process according to claim 46, wherein the solubility enhancer is preferably Polyvinyl pyrrolidone.

48. The process according to claim 46, wherein the co-solubilizing agent is preferably Propylene glycol.

49. The process according to claim 46, wherein the solubilizing agent is preferably Diethylene glycol monoethyl ether.

50. A process for producing pharmaceutical composition as claimed in claim 1, comprising:
adding solubility enhancer & solubilizing agent Ethyl alcohol to co-solubilizing agent & mixing; adding Ibuprofen sodium dihydrate to the mixture & further mixing; and
disposing resulting solution in soft gelatin capsules.

51. The process according to claim 50, wherein the solubility enhancer is preferably Propylene glycol.

52. The process according to claim 50, wherein the solubilizing agent is preferably Ethyl alcohol.

53. The process according to claim 50, wherein the co-solubilizing agent is preferably Polyethylene Glycol 400.
